(19) United States
(12) Patent Application Publication
(10) Pub. No.: US 2008/0228226 A1
(43) Pub. Date: Sep. 18, 2008

(54) CERVICAL SUPPORT SYSTEM

(76) Inventor: Arya Nick Shamie, Los Angeles, CA (US)

Correspondence Address: LAW OFFICES OF KAMRAN FATTAHI, 15303 VENTURA BLVD., SUITE 1400, SHERMAN OAKS, CA, 91403, US

(21) Appl. No.: 12/047,313

(22) Filed: Mar. 12, 2008

Related U.S. Application Data
(60) Provisional application No. 60/906,272, filed on Mar. 12, 2007.

(51) Int. Cl. A61B 17/58; A61B 17/56; A61B 17/08

(52) U.S. Cl. 606/246; 606/301; 606/151

(57) ABSTRACT

An apparatus for connecting between an upper cervical vertebrae and a cervical fixation network that includes a screw for rotationally entering and gripping the upper cervical vertebrae. A bridge is configured to route between the cervical fixation network and the screw. A connector holding the screw and bridge together includes a portion of the screw and the bridge that is configured differently for a complementary, low profile engagement of the bridge and screw in which varying configurations of the connector and bridge, either alone, or in combination, are envisioned.

### CROSS-REFERENCE TO RELATED APPLICATION(S)

This application claims the benefit of U.S. Provisional Application Ser. No. 60/906,272 filed on Mar. 12, 2007, which is incorporated herein by reference.

### FIELD OF THE INVENTION

This invention relates generally to cervical support devices, and, more particularly, to a system for providing low profile cervical support to the vertebrae.

### BACKGROUND OF THE INVENTION

With the advent of modern surgical techniques, methods and systems using rigid cervical support devices have been developed to manage instability of the upper cervical spine in a human body. Such methods and systems have been implemented successfully in patients with cervical disorders requiring stabilization resulting in improved spinal support for the neck and head as well as improvements in relief from pain resulting from such instability.

One drawback to systems providing sub-axial cervical spine fixation is stabilization support for certain upper vertebrae, referred to as the C1 and C2 vertebrae. Until recently, common techniques for the placement of screws in the C2 for fixation to a fixation network such as a lateral mass screw and rod fixation system resulted in significant risks to the vertebral artery. These risks were reduced by the discovery of techniques for the unique placement of screws in the C2 for connecting the C2 to the rigid cervical rods. A description of C2 fixation problems and a technique of the type suitable for solving such problems is disclosed in “Posterior C2 Fixation Using Bilateral, Crossing C2 Laminar Screws” by Neil M. Wright, MD in the Journal of Spinal Disorders & Techniques, Vol. 17, No. 2 (April 2004), which is incorporated herein by reference.

While fit for their intended purpose, one problem created by such C2 fixation techniques is that the location and angle of the screw entry and alignment is not well suited for screw insertion and attachment to a rigid cervical rod using existing rigid cervical fixation hardware during surgery.

Thus, the need exists for a system and method to connect the C2 vertebrae to a fixation schema using the improved techniques that reduce risk to the vertebral artery that have been identified above.

### SUMMARY OF THE INVENTION

The present invention relates to an apparatus for connecting between an upper cervical vertebrae and a cervical fixation network that includes a screw having a portion with a tapered shaft with a helical rim for rotationally entering and gripping the upper cervical vertebrae. A bridge is configured to route between the cervical fixation network and the screw. A connector holding the screw and bridge together includes a portion of the screw and the bridge that is configured differently for a complementary, low-profile engagement of the bridge and screw.

The invention further includes a device for connecting to an upper cervical vertebrae including a screw having a portion with a tapered shaft threaded for rotationally entering and gripping the upper cervical vertebrae. The screw includes a connector portion for directly contacting a strip routed to a cervical fixation network to provide a low profile connection.

In another aspect of the invention, a device is included for connecting between a post anchored in an upper cervical vertebrae and a cervical fixation network. The device includes a bridge having at least one end configured for complementary connection to a post. The bridge includes at least one portion that adjusts relative to the post for routing of the bridge between the post and the cervical fixation network.

In yet another aspect of the invention, a connector for use in a cervical fixation device includes a base configured to receive at least two rods in a locking engagement. The base includes at least one channel having an aperture to receive at least one rod at any location along a shaft of the rod and a set screw fastened into the base to hold the at least two rods against the base.

### BRIEF DESCRIPTION OF THE DRAWINGS

Other aspects, advantages and novel features of the invention will become more apparent from the following detailed description of the invention when considered in conjunction with the accompanying drawings wherein:

FIG. 1 is a diagram incorporating functional block for certain structures of a screw with a connector and bridge according to the present invention;

FIG. 2A is a top plan view of a screw according to the present invention;

FIG. 2B is a side view of the screw in FIG. 2A according to the present invention;

FIG. 2C is an exploded perspective side view of a screw with a connector according to the present invention;

FIG. 3A is a top plan view of a screw according to the present invention;

FIG. 3B is a side view of the screw in FIG. 3A according to the present invention;

FIG. 4A is a top plan view of a screw according to the present invention;

FIG. 4B is a side view of the screw in FIG. 4A according to the present invention;

FIG. 5 is a side view of a bridge according to the present invention;

FIG. 6 is a side view of a bridge according to the present invention;

FIG. 7 is a side view of a bridge according to the present invention;

FIG. 8 is a side view of the screw with connector of FIG. 2C and bridge of FIG. 6 connected between a C2 vertebrae and a fixation network according to the present invention;

FIG. 9A is a front view of an alternate screw and bridge configuration according to the present invention;

FIG. 9B is a side view of the screw and bridge of FIG. 9A with a different rotational displacement relative to FIG. 9A according to the present invention;

FIG. 10A is a side view of a connector according to the present invention; and

FIG. 10B is a front view of the connector of FIG. 10A according to the present invention.

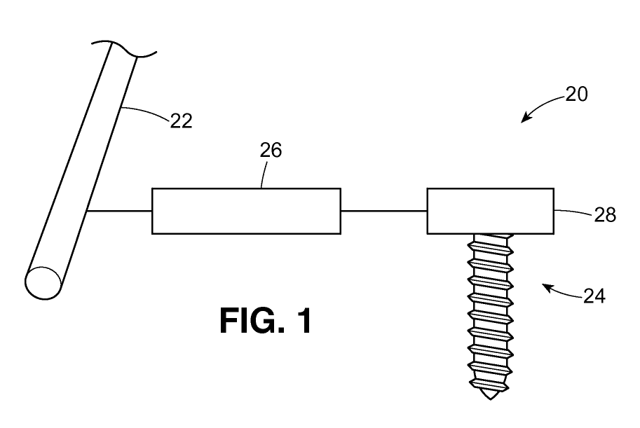

### DETAILED DESCRIPTION

With reference to the drawings for purposes of illustration, an improved system 20 is provided for fixation of the C2 to a rigid cervical fixation network 22 such as, but not limited to, a screw and rod system, using a screw or post 24. Advantageously, a low profile bridge 26 is provided between the rigid cervical fixation network 22 and the screw 24. Furthermore, a connector 28 is included that facilitates connection of the screw 24 to the bridge 26. Presently the screw 24, connector 28 and the bridge 26 may be made from any material or material combination, without limitation, suitable for insertion into a living body. For example, but not by means of limitation, the materials may include stainless steel, a cobalt/chrome alloy, titanium or any alloy combination thereof. Presently, titanium alloy materials are preferred in the medical community and therefore, for that reason, would be preferred in this invention. However, changes in materials preferred by the medical community may be substituted so long as those substitutions conform to the preferred features of the invention described below, including, but not limited to, strength of the screw to withstand rotational torque into a bone mass and strength of the combination of the screw with connector and the bridge to provide the desired amount of cervical fixation.

C2 screw and Connector

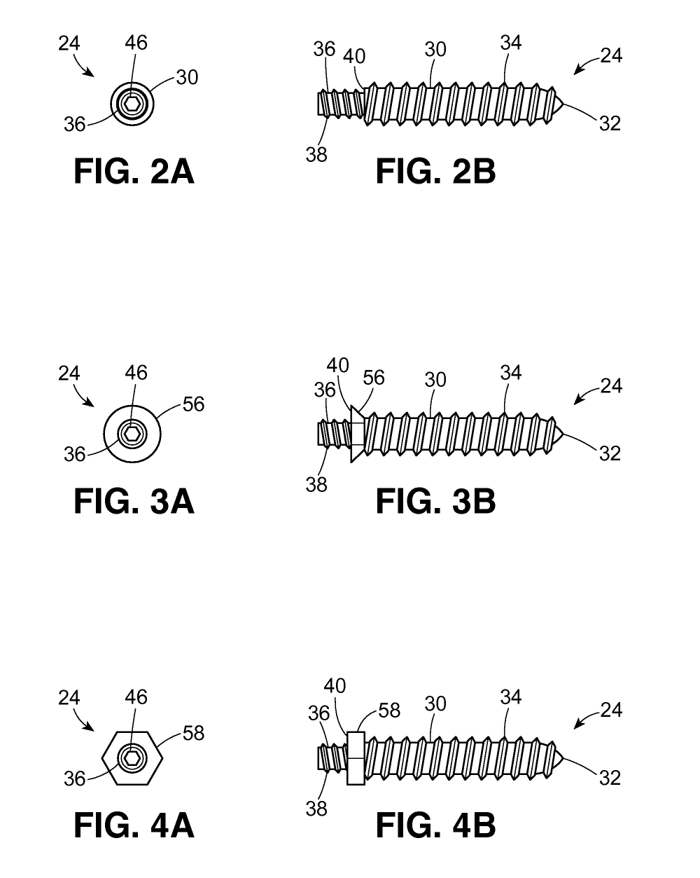

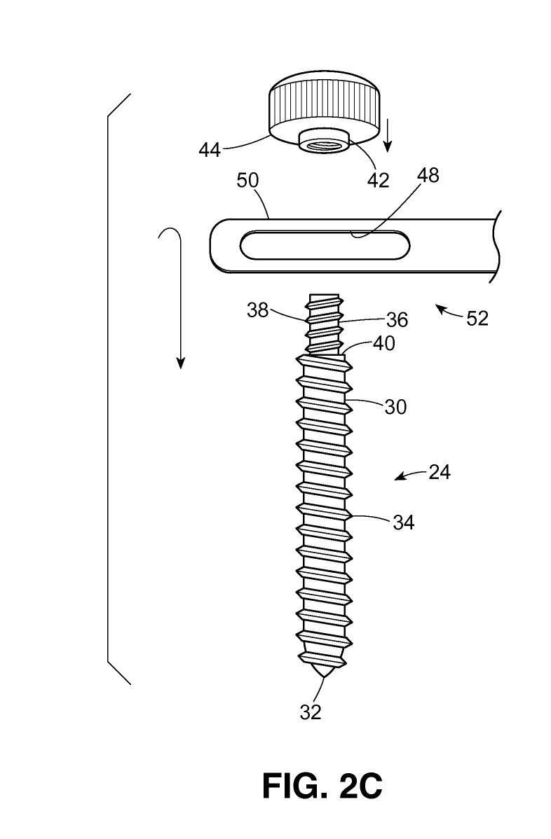

With reference to FIGS. 2A-C, the screw 24 is a threaded fastener that includes a shaft 30, cylindrical and tapering to a point 32 at one end with a helical ridge 34 or thread formed on it. The helical ridge is of the self-tapping type for rotational insertion into the C2 vertebrae. The shaft diameter narrows to form a shaft connector portion 36, cylindrical and non-tapering with a helical ridge or thread 38 formed on it. A ledge or ridge 40 is formed at the point along the shaft where the wider tapered shaft transitions to the non-tapered head shaft portion 36. The helical ridge 38 of the shaft connector portion 36 is of a non-self tapping type for insertion into an aperture 42 of a nut 44 (FIG. 2C) preformed with a complementary cylindrical wall having a helix. The free end of the shaft connector portion (FIG. 2A) is shaped to allow locking engagement 46 with a fastening tool such as a screwdriver or wrench. As presently illustrated in FIG. 2A, a hexagonal socket 46 for receiving a hex wrench is shown which permits rotation of the shaft causing the tapered portion of the shaft to be received in the C2 vertebrae. At the region of the ridge 40, the ridge preferably has radial width of 0.5 mm where the shaft of the shaft connector portion 36 has a radial diameter of 1 to 2 mm and the shaft of the tapered portion 30 at the ridge has a diameter of 3 to 4 mm. The length of the screw may vary according to the size of the C2 vertebrae in which the screw is to be inserted.

The shaft connector portion 36 has a length sufficient to receive a wide slotted aperture 48 from a screw connector portion 50 of a strip 52 comprising a portion of the bridge about the narrow diameter of the shaft connector portion 36 that rests upon the ledge 40 of the shaft. The wide slotted aperture 48 extends along the length of the metal strip 52 for a length sufficient to allow positional adjustments between the metal strip 52 and the screw 24. The nut 44 may include a locking device. Locking devices of the type suitable for this purpose may include, but are not limited to, nylon lock nuts, a serrated-face nut, a nut with a lock washer, such as a star washer, locking adhesives, a castellated nut with a pin, a split beam lock nut or any combination thereof. The nut 44 may be capped and may include an outer surface for tightening such as, but not limited to, a polygonal circumference for tightening by a wrench or a serrated face for gripping and tightening by hand or pliers.

With reference to FIGS. 3A-B, where like structures to FIGS. 2A-C incorporate the reference numerals above, a screw 24 having a tapered shaft portion 30 and a non-tapered shaft portion 36 includes a wider region 56 that tapers quickly from the ridge 40 to the tapered shaft diameter providing a conical outline similar to the bell of a bugle. The wider region 56 allows for the ridge width range to vary from 0.5 to 1 mm and for the range of the shaft connector portion 36 diameter to include a range of 1 to 4 mm. It should be noted that the wider region may be non-tapered and cylindrical without departing from the present invention.

With reference to FIGS. 4A-B, where like structures to FIGS. 2A-C incorporate the reference numerals above, a screw 24 having a tapered shaft portion 30 and a non-tapered shaft portion 36 includes a wider region 58 that extends between the two shaft portions providing a non-tapered outline having a polygonal shaped circumference that accommodates a rotational torque tool such as a wrench. For purposes of illustration only, a hexagonal shape is shown. The wider region 58 allows for the ridge width range to vary from 0.5 to 1 mm and for the range of the shaft connector portion 36 diameter to include a range of 1 to 4 mm. It should be noted that in this embodiment, the locking engagement 46 at the head of screw may be optional.

Regardless of the embodiment preferred for a particular use, each of the screws described provides a threaded fastener for secure fixation to a bone mass. The connector features a low profile solution for fixation of the screw to a rigid cervical fixation network. Furthermore the connector configuration permits adjustment of the positional relationship between the bridge and the screw.

Bridge

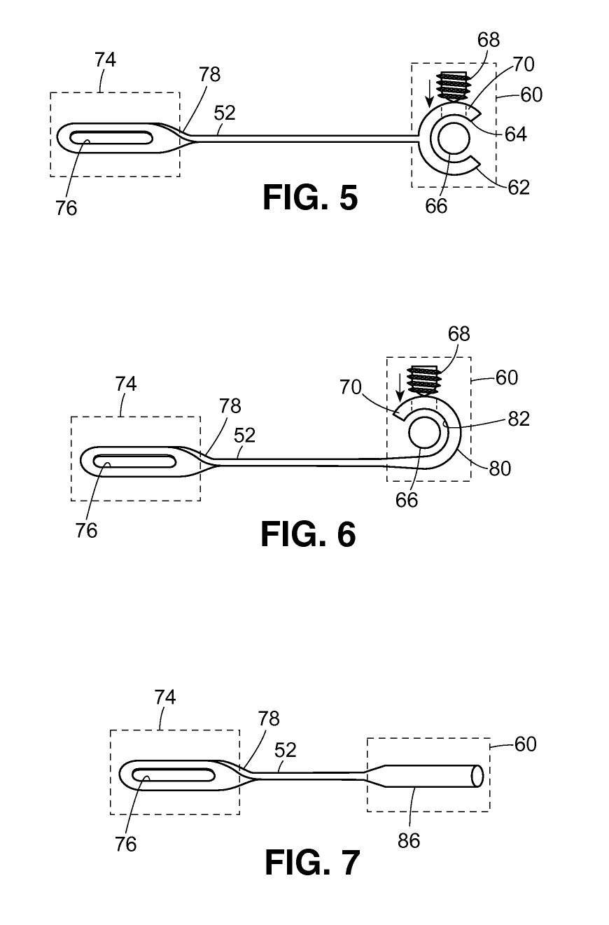

With reference to FIG. 5, a bridge includes a plate or strip 52 in which the length of the bridge 26 is adapted for a general range of distances for connection between a C2 screw 24 and a rigid cervical fixation network 22. The bridge width and thickness are proportionally configured in respective sizes according to criteria such as the modulus of elasticity of the material used, the forces received on the material when installed to provide fixed support under such forces in a fixation network, a degree of flexibility when a threshold of force is exceeded through manual pressure applied by a user during insertion to conform the plate to a compatible mating with each of the screw and the fixation network. The plate or strip 52 includes a fixation network connector 60 at one end in the form of a C-shaped sleeve 62 with a semi-cylindrical interior wall 64 with an opening for receiving a rod 66 from a fixation network. The opening of the sleeve faces out away from the opposite end of bridge. The C-shaped sleeve includes a rigid connection to the rod. The rigid connection may be accomplished by any rigid connection means suitable for a permanent rigid connection, which by way of example and not by limitation, can be by crimping or bending by force of the sleeve 62 onto the rod 66 or secured by a set screw 68 inserted through a bore hole 70 in the C-shaped sleeve 62 for rigid fixation of the rod between the screw and the C-shaped wall. A the opposite end of the bridge the strip includes the screw connector portion 74 having a wide slotted aperture 76 sized and shaped such that it is elongated along the length for spatial displacement of the bridge 26 relative to the screw 24 and dimensioned such that opposing sides the strip about the aperture may rest upon the ridge 40 (FIG. 4C) which snuggly receives the shaft connector portion 36 of the screw 24 there between. As presently illustrated, the bridge includes a generally 90-degree twist 78 (FIG. 5) in the strip 52 to demonstrate in this written description that the region between the ends may be twisted and bent to route the bridge 26 from the screw having an angle of insertion at a point in the C2 vertebrae to a fixation network having a different angle and for providing a low profile path there between the contours of the bone masses along the route. The single 90-degree angle illustrated in the application is merely to facilitate understanding and illustration of the ends of the bridge while demonstrating the ability of the structure to be bent.

With reference to FIG. 6, where like structures to FIG. 5 incorporate the reference numerals above, the plate or strip 52 includes a fixation network connector 60 at one end in the form of a C-shaped sleeve 80 with a semi-cylindrical interior wall 82 with an opening for receiving a rod 66 from a fixation network. The opening of the sleeve faces inward toward the opposite end of bridge. The C-shaped sleeve 80 includes a rigid connection to the rod. The rigid connection may be accomplished by any rigid connection means suitable for a permanent rigid connection, which by way of example and not by limitation, can be by crimping or bending by force of the sleeve 80 onto the rod 66 or secured by a set screw 68 inserted through a bore hole 70 in the C-shaped sleeve 80 for rigid fixation of the rod 66 between the screw 68 and the C-shaped wall 82.

With reference to FIG. 7, where like structures to FIG. 5 incorporate the reference numerals above, the plate or strip 52 includes a fixation network connector 60 at one end in which the strip transitions from a generally rectilinear cross-sectional shape to a generally cylindrical shape to form a rod 86 having a diameter sized to conventional rod diameters of a fixation network. The rod shape then may be connected to the fixation network using a fixation network connector for adding a rod to a fixation network.

It will further be appreciated that other configurations for the fixation network connector may be used without departing from the features of the present invention. Furthermore, the bridge may span between to screws in which the opposite ends of the bridge may include screw connector portions 74 at each end.

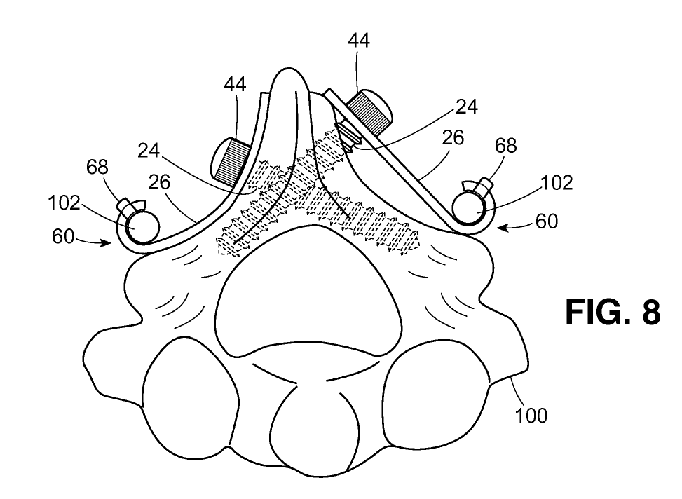

When in use, an example (FIG. 8) of a screw 24, a connector and bridge 26 is shown for attachment between a C2 vertebrae 100 and rod 102 of a fixation network 22 having two rods traversing the C2 vertebrae 100 and running the along the length of the spine for possible connection of the C2 vertebrae 100 to other vertebrae (not shown) as medically desired for the patient's medical needs. The embodiment of the screw 24 as shown in FIG. 2C and the embodiment of the bridge 26 as shown in FIG. 6 are used for illustration purposes only, but where like reference numerals of like structures are used herein. Each of the screws 24 is entered into the lamina 104 portion of the C2 vertebrae 100 from opposing angled sides using conventional insertion techniques. As shown in this configuration there is no rotational configuration of the bridge 26 required. The fixation network connector 60 in this exemplary embodiment is made by both crimping and using the setscrew 68 to hold each bridge 26 rigidly with respective rods 102. It is understood by one of ordinary skill in the art of fixation network insertion that any combination of the above described screws and bridges may be used to accomplish this connection. It is further understood by those skilled in the art of inserting fixation networks in a living body that the cervical structures encountered in a living body may vary and that screws and bridges of varying embodiments and dimensions within the ranges and lengths described may be provided and used as needed medically to make the connection.

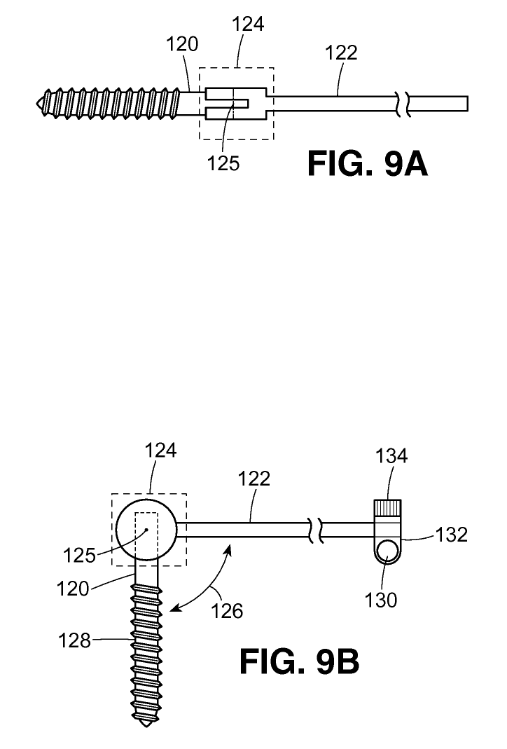

With reference to FIGS. 9A-B, an alternative embodiment of a screw 120 and bridge 122 in the form of a rod in which the connector 124 is hingedly attached together by a hinge pin 125 to allow for the angle 126 of a thread tapered shaft portion 128 of the screw 120 to be adjusted relative to the angle of the bridge 122. This configuration reduces the profile of the connector 124 at the screw 120 and allows for the bridge 122 to be angled appropriately after insertion of the screw 120 for routing to a fixation network, in which the rod shape of the bridge 122 is sized to conform with existing rod configurations for connection of the rod to a fixation network using connectors. As shown for purposes of illustration, the bridge 122 may be connected to another rod 130 using a connector 132 for two rods using a setscrew 134. However, it will be appreciated by those skilled in the art that any of the connector configurations described above may be used herein without departing this invention. It will further be appreciated that this embodiment allows for the rotationally hinged connection between the screw and the bridge to operate as a manual insertion tool to facilitate rotational entry of the screw in the C2 vertebrae and at recommended location and insertion angle by bending of the hinge sufficiently to use the leverage of the bridge to achieve the desired rotational movement.

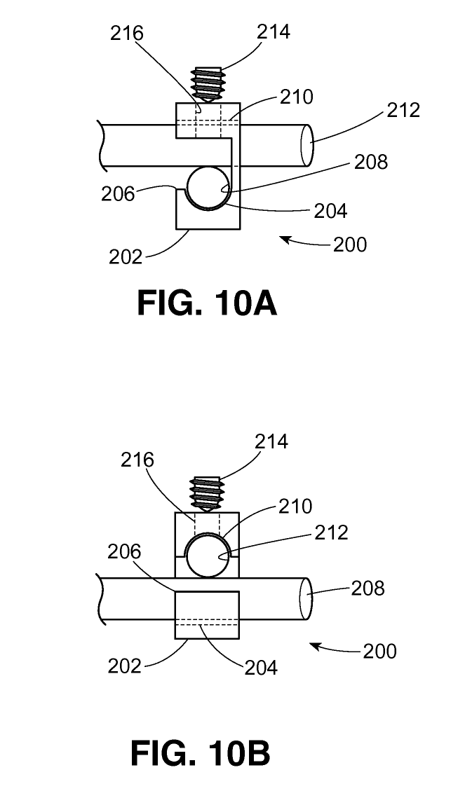

With reference to FIGS. 10A-B, a connector 200 formed to connect two rods traversing at generally 90 degrees includes generally base 202 having a seat 204 forming a channel with a C-shaped opening 206 for receiving a first rod 208 there through. The seat 204 is presently preferred to be, but not limited to, concave in cross-section. An aperture 210 allows for a second rod 212 to slide through the connector at an angle offset from the first rod. As presently illustrated the aperture 210 is generally perpendicular to the seat 204; however, the angular displacement of the aperture 210 relative to the seat 204 may vary and a plurality of connectors allowing for rods to intersect at various angles may be used. The first and second rods are held fast by the connector using a setscrew 214 inserted through a borehole 216 which the compresses the two rods 212 and 208 together between the setscrew 214 and the seat 204. It will further be appreciated that the shape of the rods may be varied to increase the fastening between them by for example including a flat surface about at least a portion of the circumference such that when the two flat surfaces overlie each other the surface area in contact increases the amount of force required to move the rods relative to each other and the connector.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is:

1. An apparatus for connecting between an upper cervical vertebrae and a cervical fixation network comprising:
a screw having a portion with a tapered shaft with a helical rim for rotationally entering and gripping said upper cervical vertebrae;
a bridge configured to route between said cervical fixation network and said screw; and
a connector including a portion of said screw and said bridge that is configured for a complementary, low-profile engagement of said bridge and screw.

2. The apparatus of claim 1 wherein said connector is a complementary tongue and groove connection between said screw and bridge couple for rotational displacement about a hinge pin.

3. The apparatus of claim 1 wherein:
said bridge includes an aperture; and
said screw includes a connector portion sized to pass through said aperture.

4. The apparatus of claim 3 wherein:
said connector includes a nut threadingly received on said screw to hold said bridge onto said screw.

5. The apparatus of claim 3 wherein said aperture is wider than said screw to allow positional displacement of said bridge relative to said screw.

6. A device for connecting to an upper cervical vertebrae comprising:
a screw having a portion with a tapered shaft threaded for rotationally entering and gripping said upper cervical vertebrae;
said screw includes a connector portion for directly contacting a strip routed to a cervical fixation network to provide a low profile connection.

7. The device of claim 6 wherein:
said tapered shaft portion has a cross-sectional diameter different in size than a cross-sectional diameter of said connector portion;
such that a ridge is formed at a location where said tapered shaft portion and said connector portion meet.

8. The device of claim 6 wherein:
said connector portion is not tapered.

9. The device of claim 6 wherein:
said connector portion includes a free end having a shape to form a locking engagement with a fastening tool.

10. The device of claim 6 including:
a nut having a bore hole to be received by said connector portion of said screw.

11. The device of claim 10 wherein:
said nut includes a locking device.

12. The device of claim 11 wherein:
said locking device is selected from the group consisting of a nylon locking nut, a serrated-face nut, a nut with a lock washer, a star washer nut, locking adhesive, a castellated nut and pin, a split beam lock nut and any combination thereof.

13. The device of claim 11 wherein:
said nut is capped.

14. The device of claim 11 wherein:
said nut includes an outer surface shaped for gripping during rotational tightening.

15. A device for connecting between a post anchored in an upper cervical vertebrae and a cervical fixation network comprising:
a bridge having at least one end configured for complementary connection to a post;
at least one portion of said bridge that adjusts relative to said post for routing of said bridge between said post and said cervical fixation network.

16. The device of claim 15 wherein:
said bride includes an aperture sized to receive said post there through.

17. The device of claim 16 wherein:
said aperture is wider than said port to allow displacement of said post relative to said bridge.

18. The device of claim 15 wherein:
said bridge is formed from a strip of implantable surgical material.

19. The device of claim 18 wherein:
said strip is formed from the group consisting of titanium, cobalt, chrome, stainless steel and any combination thereof.

20. The device of claim 18 wherein:
said bridge may be bent to contour a bone.

21. The device of claim 15 wherein:
said bridge includes a network connector.

22. The device of claim 15 wherein:
said bridge includes a metal strip portion and a rod portion.

23. The device of claim 15 wherein:
said bridge includes a C-shaped portion having an aperture to receive a rod.

24. The device of claim 23 wherein:
said rod is fastened to said bridge.

25. The device of claim 24 wherein:
said fastener is selected from the group consisting of crimping said C-shaped portion, a set screw and any combination thereof.

26. The device of claim 23 wherein:
said aperture is outwardly facing.

27. The device of claim 23 wherein:
said aperture is inwardly facing.

28. A connector for use in a cervical fixation device comprising:
a base configured to receive at least two rods in a locking engagement;
said base including at least one channel having an aperture to receive at least one rod at any location along a shaft of said rod; and
a set screw fastened into said base to hold said at least two rods against said base.
